(19) United States
(12) Patent Application Publication
(10) Pub. No.: US 2008/0210771 A1
(43) Pub. Date: Sep. 4, 2008

(54) Two-Substance Atomizing Device

(75) Inventors: Nikolaos Zarzalis, Karlsruhe (DE); Lambert Krebs, Stutensee (DE); Emmanouil Pantouflas, Karlsruhe (DE); Hans-Joachim Wierner, Eggenstein-Leopoldshafen (DE)

Correspondence Address: DARBY & DARBY P.C., P.O. BOX 770, Church Street Station, New York, NY, 10008-0770, US

(73) Assignee: FORSCHUNGSZENTRUM KARLSRUHE GMBH, Karlsruhe (DE)

(21) Appl. No.: 12/064,282

(22) PCT Filed: Jul. 27, 2006
(86) PCT No.: PCT/EP2006/007432

§ 371 Date: Feb. 20, 2008

(30) Foreign Application Priority Data
Aug. 20, 2005 (DE) 10 2005 039 412.4

(51) Int. Cl. A61L 9/04

(52) U.S. Cl. 239/58

(57) ABSTRACT

A two-substance atomizing device for atomizing a fluid in a gas stream includes: a fluid supply branch including a nozzle orifice; a gas supply branch configured concentrically about the fluid supply branch; a concentric second gas branch branching from the gas supply branch and disposed upstream of the nozzle orifice, the nozzle orifice terminating in the gas supply branch; an axially symmetric mixing channel disposed in axial extension of the fluid supply branch and including a discharge orifice; an annular injection orifice in communication with the gas supply branch and disposed near the discharge orifice; and an adjustment device configured to adjust a cross section of the injection orifice.

### CROSS-REFERENCE TO RELATED APPLICATIONS

This is a U.S. national phase application under 35 U.S.C. § 371 of International Patent Application No. PCT/EP2006/007432, filed Jul. 27, 2006, and claims benefit of German Patent Application No. 10 2005 039 412.4, filed Aug. 20, 2005. The International Application was published in German on Mar. 1, 2007 as WO 2007/022847 A1 under PCT Article 21(2).

### FIELD

The present invention relates to a two-substance atomizing device for atomizing a fluid in a gas stream.

### BACKGROUND

To meet the stringent requirements of atomization with respect to spray quality, flow rate and control range, gas-based atomizers, which are also referred to in the following as two-substance atomizing devices, are used. In this context, the energy required for atomizing the fluid is introduced by the gas (air, vapor or inert gases), i.e., as a function of the relative velocity between the gas and fluid. Typically, the fluid is injected directly into the gas stream and atomized by the gas flow.

The aforementioned atomizer group also includes internal-mixing two-substance atomizing devices having a coaxial fluid feed. For the most part, these devices are characterized by an axially symmetric atomizer design and have a mixing channel disposed therein into which the fluid supply and the gas supply lead and which, in turn, has a discharge orifice to the ambient surroundings. The atomized fluid-gas mixture exits the discharge orifice as a finished product, so that these devices differ from systems where the atomization takes place externally to the device (as in the case of paint spray guns, for instance). Typical examples of the internal-mixing two-substance atomizing device include burners for internal combustion systems, such as heavy oil burners, or prechamber Diesel engines (prechamber=mixing channel).

The large number of influencing parameters and the lack of information on the spray characteristic prevent satisfactory optimization of the two-substance atomizing device. One of the most significant technical problems encountered when working with internal-mixing atomizing devices in use today is the formation of film on the wall of the mixing channel (channel wetting). Certain operating environments entail the probability of the fluid jet widening too quickly within the mixing channel inside of the atomizing device and thus of a wall film forming within the discharge channel. This film is pressed by the flow of the gas-fluid mixture toward the discharge orifice, where it is entrained by the flow as larger drops. In this respect, efforts are directed to providing a gas flow state directly at the discharge orifice that not only entrains the drops, but also atomizes the same. One of these gas-flow states is realized by injecting a sheathing gas for the discharging gas-fluid mixture.

The German Patent Application DE 199 41 091 A1 describes exemplarily a heating oil burner having an internal-mixing, two-substance atomizer nozzle which is provided with an additional, annular gas injection at the outlet leading out from the mixing channel.

The German Patent Application DE 35 25 161 A1 also describes a correspondingly designed mixing channel outlet of a device for atomizing the liquid fuels or high-viscosity fuels.

However, the aforementioned devices do not provide for any direct control of the injection orifices that would allow, in particular, the nozzle geometry to be modified during operation, i.e., without interrupting the atomization process.

Under known methods heretofore, the geometric parameters of two-substance atomizing devices with or without injection have been set in relation to an operating condition (gas to fluid ratio, Weber number, the Ohnesorge number). However, this entails the significant drawback that the atomizer nozzle designed in this manner is also tied to the particular operating conditions. Therefore, it may be that the internal-mixing two-substance atomizing devices that are used do not exhibit any wetting of the mixing channel (wall film formation) for these operating conditions; however, they are also limited in their practical use. Thus, their range of application is greatly dependent on the operating conditions. Variations in the operating conditions, changes in the substance properties, or the use of a different fluid may very quickly render a particular nozzle unsuited for an atomization process.

### SUMMARY

An aspect of the present invention is to provide a two-substance atomizing device which has universal applicability, does not exhibit the aforementioned limitations and, in particular, which is also adaptable to changes in basic conditions during a continuous operation, such as the device temperature at the mixing channel wall.

In an embodiment the present invention provides a two-substance atomizing device for atomizing a fluid in a gas stream. The device includes: a fluid supply branch including a nozzle orifice; a gas supply branch configured concentrically about the fluid supply branch; a concentric second gas branch branching from the gas supply branch and disposed upstream of the nozzle orifice, the nozzle orifice terminating in the gas supply branch; an axially symmetric mixing channel disposed in axial extension of the fluid supply branch and including a discharge orifice; an annular injection orifice in communication with the gas supply branch and disposed near the discharge orifice; and an adjustment device configured to adjust a cross section of the injection orifice.

### BRIEF DESCRIPTION OF THE DRAWINGS

Aspects of the present invention will now be described by way of exemplary embodiments with reference to the following drawings, in which:

FIG. 1 is a lateral sectional view of an exemplary embodiment of the present invention; and

FIG. 2 is a lateral sectional view of another exemplary embodiment of the present invention.

### DETAILED DESCRIPTION

An aspect of the present invention provides the ability to adjust the geometry, i.e., cross section, of the injection orifice in a controlled process, and thus the design thereof in terms of fluid mechanics. This includes the radial cross section and/or the axial cross section (flow channel profile), the concept of controlled adjustability applying primarily to modifications to the injection orifice. Within the context of the present invention, also included in the concept of adjustability are the measures used for changing the flow conditions per se which are modifiable as well by the arrangement used for that purpose, even when they do not directly affect the nozzle orifice. This also includes a change in the flow conditions (for example, turbulent and laminar flow regions, reverse flows, etc.) that occur at the gas branching-off areas or in response to specific deflections. These variations occur naturally along with the back pressure conditions and are adjustable as a function of the geometry of the nozzle orifice. Depending on the specific embodiment, the channel cross section likewise varies not only at the injection orifice, but also in the gas flow control upstream of the injection orifice.

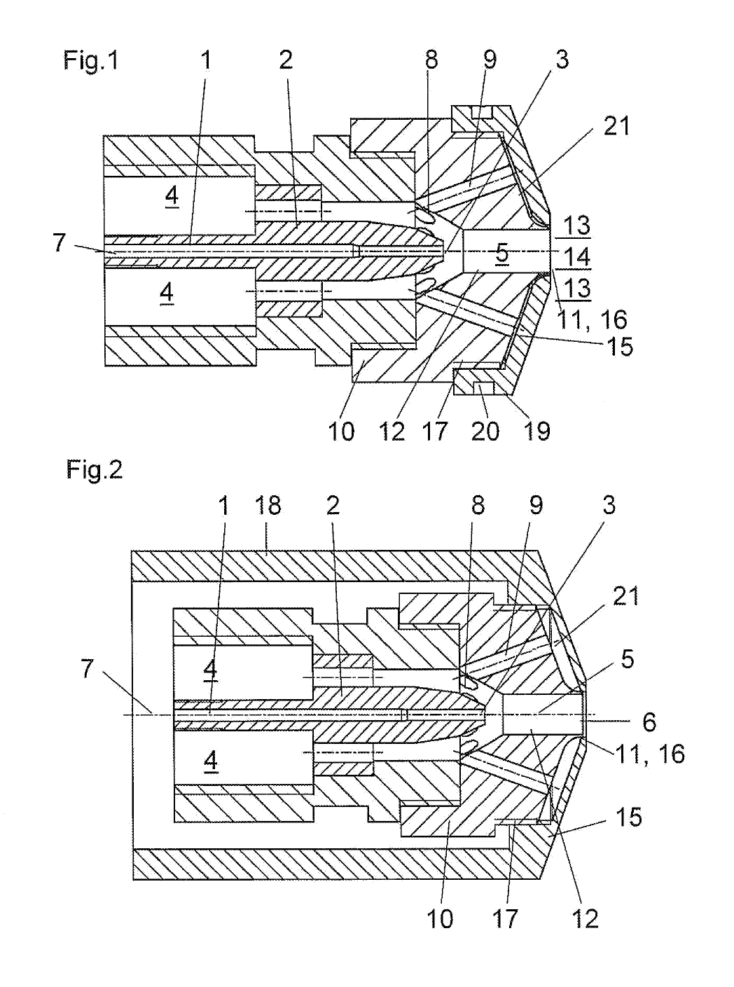

Both exemplary embodiments of the two-substance atomizing device include a fluid supply 1 in a preferably tubular fluid-supply housing 2 having a (distal-side) nozzle orifice 3 and a gas supply 4 configured concentrically about the fluid supply, respectively the fluid-supply housing, the nozzle orifice terminating in the gas supply. Disposed downstream (distally) in the extension of this substance convergence is mixing channel 5 in which the aforementioned substances (gas and fluid) mix and exit again via a (distal-side) discharge orifice 6. Fluid supply 1, gas supply 4, mixing channel 5 and discharge orifice 6 are preferably axially symmetric about a bisecting line 7. Also provided is a gas branch 8, which is arranged concentrically to the aforementioned components and which branches off from gas supply 4 prior to injection of the fluid, i.e., before reaching nozzle orifice 3, and which leads into an annular injection orifice 11 at discharge orifice 6. In FIGS. 1 and 2, the gas branches are implemented, for example, by a multiplicity of bores 9 arranged concentrically about bisecting line 7 within mixing-channel housing 10. Injection orifice 11 leads axially and preferably at an acute angle, i.e., to the extent that it is technically feasible, in parallel to mixing-channel wall 12, directly at and around discharge orifice 6, resulting in a peripheral flow 13 around atomized substance mixture 14 that emerges via the discharge orifice (compare FIG. 1), thereby entraining the fluid film precipitated onto mixing-channel wall 12.

The adjustment device includes a sleeve nut or threaded cap 15 having a bore 16 that is adjustable by a relative rotation on a thread 17 on mixing-channel housing 10.

FIG. 1 illustrates an arrangement whose threaded cap 15, at peripheral surface 19 thereof, has a profiled section 20 that is movable and fixable with form locking engagement. The profiling is preferably implemented by a gear-tooth system, in the operating state, the gear-tooth system either being driven by a chain, belt or gear wheel arrangement (not shown) of a positioning drive that engages with form locking on the profiled section. In principle, frictionally engaging positioning drives also being suited, it being necessary, however, for the frictional engagement to be ensured for every occurring operating state.

FIG. 2 shows exemplarily arrangement whose threaded cap 15 includes a housing 18 for all other components (1 through 14) of the two-substance atomizer nozzle. An adjustment is performed during operation by applying torsion either to the aforementioned components of the two-substance nozzle within fixed housing 18 or to housing 18 on thread 17 of the otherwise fixed two-substance atomizer nozzle (components (1 through 14)).

Discharge orifice 11 may be coplanar to bore 16 (as in FIG. 1, for example) or be offset from the same (compare FIG. 2), a change in these two states, which also have a determinative effect on the inflow direction, being made possible by the aforementioned arrangement during operation.

An aim of the two-substance atomizing device, according to an exemplary embodiment of the present invention, is to provide a field of application that is independent of the operating state. The exemplary embodiments are not provided for the purpose of avoiding wetting of the mixing-channel wall, for example by optimizing flow during atomization, which would result in a limited range of application, but rather only for eliminating wetting when it occurs as fluid drops at the discharge orifice. Here the advantage is derived that the actual atomized substance mixture is also produced using the same parameters. A basic concept of the internal-mixing two-substance atomizing device having a coaxial fluid feed is retained in the process. The central idea of the present invention is for only a smallest possible proportion of the atomization gas (approximately 10%, maximally 20%), whose volumetric flow rate is optimally adjustable during operation, to be directed upstream of the mixing channel, i.e., prior to the gas making contact with the fluid, in an annular gap 21 that is coaxial to discharge orifice 11. Externally to the two-substance atomizer nozzle, the gas emerging from the annular gap comes in contact with the film that is dripping off from the channel wall and breaks up the same. The fundamental advantage of this two-substance atomizing device resides in the atomization of the wall film occurring at the mixing-channel wall in a uniquely optimally adjustable process that is effective and, at the same time, economical.

1-5. (canceled)

6: A two-substance atomizing device for atomizing a fluid in a gas stream, comprising:
a fluid supply branch including a nozzle orifice;
a gas supply branch configured concentrically about the fluid supply branch;
a concentric second gas branch branching from the gas supply branch and disposed upstream of the nozzle orifice, the nozzle orifice terminating in the gas supply branch;
an axially symmetric mixing channel disposed in axial extension of the fluid supply branch and including a discharge orifice;
an annular injection orifice in communication with the gas supply branch and disposed near the discharge orifice; and
an adjustment device configured to adjust a cross section of the injection orifice.

7: The two-substance atomizing device as recited in claim 6, further comprising a mixing channel housing, and wherein the second gas branch includes a plurality of concentrically distributed bores disposed in the mixing channel housing.

8: The two-substance atomizing device as recited in claim 6, wherein the adjustment device includes a threaded cap adjustable by a thread rotation and having a central bore forming the annular injection orifice, the adjustment device being disposed concentrically on a thread on a mixing-channel housing.

9: The two-substance atomizing device as recited in claim 7, wherein the adjustment device includes a threaded cap adjustable by a thread rotation and having a central bore forming the annular injection orifice, the adjustment device being disposed concentrically on a thread on the mixing-channel housing.

10: The two-substance atomizing device as recited in claim 8, wherein the threaded cap includes a housing.

11: The two-substance atomizing device as recited in claim 9, wherein the threaded cap includes a housing.

12: The two-substance atomizing device as recited in claim 8, wherein the threaded cap includes a profiled section at a peripheral surface thereof and configured to be movable and fixable with a form locking engagement.
